(19) United States
(12) Patent Application Publication
(10) Pub. No.: US 2022/0398914 A1
(43) Pub. Date: Dec. 15, 2022

(54) Wearable Device and System for Tracking and Sharing Vital Signs and Location of User

(71) Applicant: Miguel Cardona, Saint Cloud, FL (US)

(72) Inventor: Miguel Cardona, Saint Cloud, FL (US)

(21) Appl. No.: 17/531,176

(22) Filed: Nov. 19, 2021

Related U.S. Application Data
(60) Provisional application No. 63/209,168, filed on Jun. 10, 2021.

(51) Int. Cl. G08B 25/01; G08B 21/02; G08B 21/08; G08B 25/12; A61B 5/00; A61B 5/0205; A61B 5/024; A61B 5/0245; G06F 1/16

(52) U.S. Cl. CPC G08B 25/016; G08B 21/0211; G08B 21/088; G08B 21/0236; G08B 21/0269; G08B 21/0277; G08B 25/12; A61B 5/0002; A61B 5/02055; A61B 5/02405; A61B 5/02438; A61B 5/02455; A61B 5/681; G06F 1/163

(57) ABSTRACT

The present invention relates to a wearable smart appliance in the form of a wristwatch. The appliance is designed to track a user's vital signs and send real-time alerts to a paired electronic device for remote monitoring of the user. More specifically, the appliance tracks vital signs such as heart rate, and notifies a parent or guardian if the user is in danger. The appliance also includes a water sensor that activates an alert if the sensor is underwater for a certain length of time. A GPS sensor detects the current location of the user and is included in the real-time alerts during emergencies.

### CROSS-REFERENCE TO RELATED APPLICATION

The present application claims priority to, and the benefit of, U.S. Provisional Application No. 63/209,168, which was filed on Jun. 10, 2021 and is incorporated herein by reference in its entirety.

### FIELD OF THE INVENTION

The present invention relates generally to the field of wearable tracking devices. More specifically, the present invention relates to a smart wearable watch that is used for alerting a user of vital signs and location. The watch includes a heart rate sensor for detecting a user's heart rate, a water depth sensor for sensing time and depth of the user in or underwater and a location sensor for detecting the location of the user. The watch transmits alerts when the values detected by the sensors do not fall within the threshold values. The watch also has a panic button for sending an alert in case of any emergency. The invention also has a software application for installing on an electronic device, and is designed to receive alerts from the watch. Accordingly, this disclosure makes specific reference thereto the present invention. Nonetheless, it is to be appreciated that aspects of the present invention are also equally applicable to other like applications, devices and methods of manufacture.

### BACKGROUND

By way of background, conventional vital sign monitors are used throughout hospitals and other medical facilities and premises. Medical professionals are required to measure the vital signs of patients in such areas and generally, the staff of medical professionals is outnumbered by the number of patients.

Currently, vital signs can be monitored via wearable devices, but they are inefficient. Especially for children and elderly people, it is important for other users and guardians to be able to check their vital signs and location without being physically present with their family members. Users desire that they should be notified immediately in cases where their children, elderly people or even adults wander off, get lost, become hurt or are in any other emergency or stressful situation. Further, vital signs accurately indicate the condition of a person in an emergency situation, in addition to a medical emergency. Therefore, users desire to remotely receive and be notified of the vital signs of their loved ones.

Further, conventional safety devices do not allow users to notify their guardians and relatives when they are medically stable but are in a dangerous situation or involved in an accident. Generally, a person has to make a call or send a message, which can be impossible in such cases. Therefore, users desire a device, preferably wearable, that can alert others of the dangerous situation. This is especially essential for children to protect them and track them in case of abduction.

Therefore, there is a long felt need in the art for a safety device for children, the elderly or users with disabilities, etc., that can be used for preventing users from getting lost, abducted or involved in accidents. There is also a long felt need in the art for an improved safety device that is capable of tracking vital signs such as a user's heart rate, and notifies parents or guardians if the user is in danger. Additionally, there is a long felt need in the art for a safety device that activates an alert to save the user from drowning. Further, there is a long felt need in the art for a wearable safety device that tracks the exact location, and notifies parents or guardians of the exact location of the user in case of a dangerous situation. Finally, there is a long felt need in the art for a safety device that prevents children, the elderly, and others from getting lost, abducted or involved in an accident by alerting their parents, guardians and others.

The subject matter disclosed and claimed herein, in one embodiment thereof, comprises a safety system. The safety system further comprises a wearable smartwatch configured for monitoring one or more vital signs. The smartwatch includes a processor, a heart rate sensor for measuring real time heart rate, a location sensor for detecting the real-time location of the user, a water depth sensor and a timer for measuring the depth and time the watch has been submerged in a body of water. Further, a smartphone having an installed computer-implemented application paired with the wearable smartwatch, and real-time alerts based on the measurements of one or more of the heart rate sensor, location sensor and water depth sensor are also provided.

In this manner, the novel wearable appliance of the present invention accomplishes all of the foregoing objectives, and provides a relatively safe, easy, and convenient solution for automatically tracking the vital signs of a user, while transmitting alerts when the vital signs are beyond threshold values. Users can be notified remotely, and a panic button transmits a sound alert for immediate rescue of the user. The appliance prevents children, the elderly, and others from becoming lost, abducted or involved in an accident by sharing the location of the user.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a safety system. The safety system comprises a wearable smartwatch configured to monitor one or more vital signs. The smartwatch includes a processor, a heart rate sensor for measuring a user's heart rate in real-time, a location sensor for detecting a user's location in real-time and a water depth sensor along with a timer for measuring depth and time the watch has been submerged in a body of water. Further, the safety system includes a smartphone having an installed computer-implemented application paired with the wearable smartwatch, which receives alerts in real-time based on the measurements of one or more of the heart rate sensor, location sensor and/or water depth sensor.

In yet another embodiment of the present invention, a wearable appliance is disclosed. The wearable appliance includes a wristwatch designed to be worn on a user's wrist. The wristwatch comprises a metal electrode at the rear surface that touches the user's skin and which measures a user's vital signs, and a housing disposed between the dial and the rear surface that houses a plurality of sensors including a heart rate sensor, a water depth sensor, a temperature sensor, a processor, a location sensor and a wireless transceiver. The wristwatch generates alerts during stressful or emergency situations, which are detected based on the measured values by said one or more sensors.

In yet another embodiment of the present invention, a smart wristwatch is disclosed. The wristwatch has a panic button that can be pressed by a user to send an emergency alert signal to one or more paired smartphones, and an alert signal to one or more emergency services, such as 911, police or medical aid services. The panic button is disposed on the dial of the wristwatch and is preferably used in stressful or emergency conditions.

In yet another embodiment of the present invention, a smart wristwatch device is disclosed. The wristwatch device includes a processor, a heart rate sensor for detecting a user's heart rate, a wireless transceiver for transmitting real-time alerts in case the heart rate is outside a safe range and a display for displaying one or more of the current time, date, vital signs and current location.

In yet another embodiment of the present invention, a smart wristwatch device for alerting others when a user of the wristwatch is drowning or struggling in water is disclosed. The wristwatch includes a location sensor for detecting the exact location of the user, a water sensor coupled to a timer for detecting the water depth and the time that the user has been in water, and a processor coupled to the water sensor for generating and transmitting an alert to one or more paired smartphones, indicating that the user is drowning or struggling, when the time that the watch is in water is greater than a predefined threshold. The wristwatch also generates a sound alert using a speaker to alert nearby people.

In yet another embodiment of the present invention, the wristwatch can use one or more of Bluetooth protocol, Wi-Fi protocol, WiMAX protocol, Cellular network or ultrawideband protocol for transmitting alerts.

In another aspect of the present invention, a safety system is disclosed that comprises a wristwatch which communicates alerts based on a user's vital signs using a wireless channel. The wristwatch includes a processor coupled to a transceiver, two or more sensors in communication with the processor to detect a user's vital sign data and a health software application executed by a second processor of an electronic device to receive said alerts. The application can also provide a health analysis of the user based on historical health data.

In another aspect of the present invention, a vital sign monitoring system for a user is disclosed and includes one or more electronic devices connected to a smart wearable watch. The smart watch has a wireless transceiver adapted to communicate with the one or more electronic devices, and a software module installed on said one or more electronic devices for receiving alerts based on a user's detected vital signs from the smart watch.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the smart wearable system of the present invention designed to alert others if a user is in a dangerous or stressful situation in accordance with the disclosed architecture;

FIG. 2 illustrates a schematic diagram showing connection of the smart wearable watch of the present invention with a companion smartphone application installed on an electronic device in accordance with the disclosed architecture;

FIG. 3 illustrates a block diagram showing exemplary components and sensors of the smart wearable watch of the present invention in accordance with the disclosed architecture;

FIG. 4 illustrates a flow diagram showing operation of the smart wearable system detecting a drowning condition and generating a drowning alert via the save life watch of the present invention in accordance with the disclosed architecture;

FIG. 5 illustrates a perspective view showing a notification sent from the smart wearable watch worn by a child in a body of water in accordance with the disclosed architecture;

FIG. 6 illustrates a flow diagram showing generation of an emergency alert by the smart wearable watch of the present invention when an abnormal heart rate is detected in accordance with the disclosed architecture;

FIG. 7 illustrates a perspective view of information displayed on one potential embodiment of the smart wearable watch of the present invention in accordance with the disclosed architecture;

FIG. 8 illustrates a flow diagram showing an alert generated and transmitted by the smart wearable watch of the present invention when a user pushes the panic button disposed on the watch in accordance with the disclosed architecture;

FIG. 9 illustrates a perspective view showing a user of the smart wearable watch of the present invention utilizing the location monitoring sensor during a child abduction in accordance with the disclosed architecture; and

FIG. 10 illustrates an exemplary electronic device upon which the software application that communicates with the smart wearable watch of the present invention is installed in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a safety device for children, the elderly or people with disabilities, etc., that can be used to prevent users from becoming lost, abducted or drowning. There is also a long felt need in the art for an improved safety device that is capable of tracking vital signs, such as heart rate, and notifies parents or guardians if the user is in danger. Additionally, there is a long felt need in the art for a safety device that activates an alert to save a user from drowning. Further, there is a long felt need in the art for a wearable safety device that tracks a user's exact location and notifies parents or guardians during emergencies. Finally, there is a long felt need in the art for a safety device that prevents children, the elderly and people with disabilities from becoming lost, abducted, etc., by alerting parents, guardians and others during emergencies.

The present invention, in one exemplary embodiment, is a wearable safety appliance. The wearable appliance includes a wristwatch designed to be worn on a user's wrist. The wristwatch comprises a metal electrode at the rear surface that touches a user's skin to measure vital signs, and a housing disposed between the dial and the rear surface that houses a plurality of sensors, including a heart rate sensor, a water depth sensor, a temperature sensor, a processor, a location sensor and a wireless transceiver, wherein the wristwatch generates alerts when an emergency situation is detected based on the measured values by said one or more sensors.

Embodiments of the present disclosure comprise computer-executable instructions, including algorithms executed by a programmable computer. However, the disclosure can be practiced with other computer system configurations as well. Certain aspects of the disclosure can be embodied in a special purpose computer or data processor that is specifically programmed, configured or constructed to perform one or more of the computer-executable algorithms described below. Accordingly, the term “computer” as generally used herein refers to any data processor and includes Internet appliances, hand-held devices including tablets, computers, wearable computers, cellular or mobile phones, multi-processor systems, processor-based or programmable consumer electronics, network computers, minicomputers, etc.

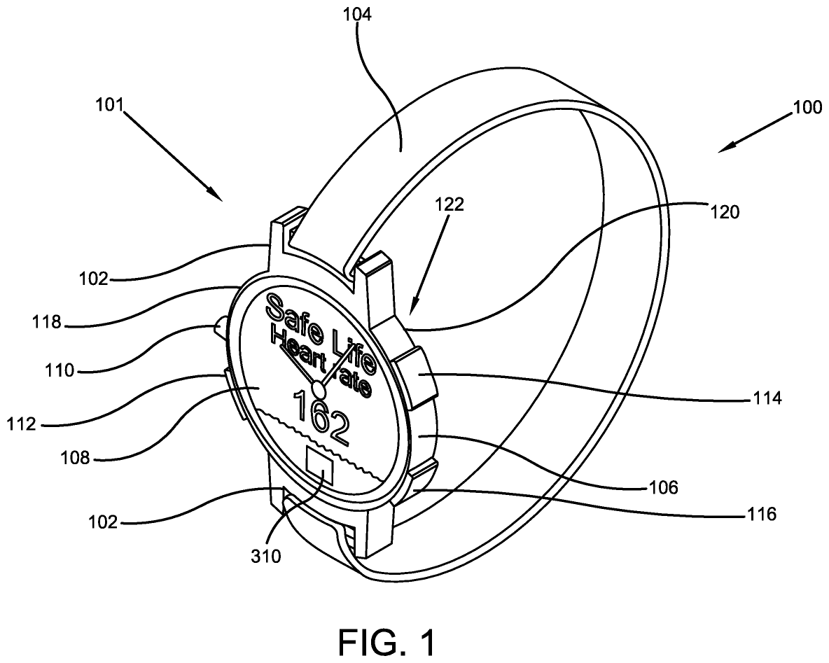

Referring initially to the drawings, FIG. 1 illustrates a perspective view of one embodiment of the smart wearable system of the present invention designed to alert others if a user is in a dangerous or stressful situation. The smart wearable system (or safety system) 100 of the present invention comprises a wearable device (or save life watch) 101, preferably in the form of a flexible wristwatch or other suitable device. The save life watch device 101 is designed to alert others when a user is in a dangerous situation such as lost, abducted by a stranger, experiencing an abnormal heart rate or drowning in a pool, lake or other body of water, etc., or any other stressful situation/emergency as is known in the art. The watch device 101 determines that a user is in a dangerous situation based on information from vital signs captured by a plurality of sensors disposed within the watch device 101.

The watch device 101 of the present invention is manufactured in various sizes, designs and configurations and can be worn by children, elderly people or adults with disabilities, etc., as well as any other suitable user as is known in the art. The watch device 101 is typically worn on the wrist using a wrist strap 104 connected via wrist buckles 102. The wrist strap 104 is replaceable and can be replaced with a new wrist strap of different design or color. The wrist strap 104 can be made of an elastic and flexible material such as silicon or rubber. Specifically, the wrist strap 104 can be made of an elastically-stretchable material such as a Thermoplastic Elastomer (TPE), a Thermoplastic polyurethane Elastomer (TPU), latex, silicone, etc., or any other suitable material as is known in the art.

The watch device 101 of the present embodiment has a round enclosure body 106 and a wrist strap 104 for securing the watch device 101 around a user's wrist. It should be noted that the body/dial 106 can be of any geometric shape as is known in the art, such as a square, rectangle, oval or any other type of shape, etc. The round enclosure body 106 has an analog display 108 shown with hour marks, but can comprise a digital display in the alternative. The watch device 101 is a smartwatch that can display the time and other vital information in digital format. Based on the preferences of the user, the display 108 can be switched from analog to digital and vice versa.

The watch device 101 has a crown 110 that is used for switching the display from digital to analog and vice versa. The crown 110 can be used for adjusting the time and date displayed on the display 108. The display 108 is also configured to display vital information such as heart rate, blood pressure and related measurements of the user. The display 108 can show various themes that can be selected by the user, based on a user's wants and/or needs. Preferably, the display 108 is a continuous display and can be made up of a Liquid Crystal Display (LCD), such as a pure reflective mode (like traditional LCD wrist watches), or with a combination of both a backlit transmission mode and a reflective mode, which is called a Transflective LCD, or any other suitable modes as is known in the art.

The watch device 101 has a plurality of control buttons on the side of the watch body 106 for operating the watch device 101, and also for alerting others about an emergency situation. For example, a panic button 112 is integrated at the side of the round enclosure body 106 and can be pressed by the user during an emergency situation, such as when abducted by a stranger. When the panic button 112 is activated, a sound alert from the watch device 101 is generated and an alert is sent to the paired smartphone. Additionally, the watch body 106 has a plurality of push buttons 114, 116 for adjusting the settings of different sensors. The push buttons 114, 116 are selectively used for different sensors disposed on the watch device 101. The watch device 101 also has a speaker 118 for emitting a sound in case of an emergency. The rear surface 120 of the watch device 101 has a metal electrode 122 that allows differential ECG or heart rate variability to be measured.

When the watch device 101 is worn on a user's wrist, the metal electrode 122 contacts the skin of the user for measuring vital signs. A differential amplifier can also be used along with the metal electrode 122, thereby allowing correct measurements to be calculated by the watch device 101 to eliminate false positives. The watch body 106 includes a PCB (Printed Circuit Board) to house a plurality of electronic components such as transducers, sensors and other circuitry, along with a battery to power the electronic components. The watch device 101 of the present invention can be charged both through a wired charger or through a wireless charger. It should be noted that all the electronic components within the watch body 106 of the watch device 101 are connected through one or more wired circuits. The wired circuits are insulated and protected from moisture and water from a swimming pool, lake or other body of water. The details of the sensors and other electronic components will be described in further figures below.

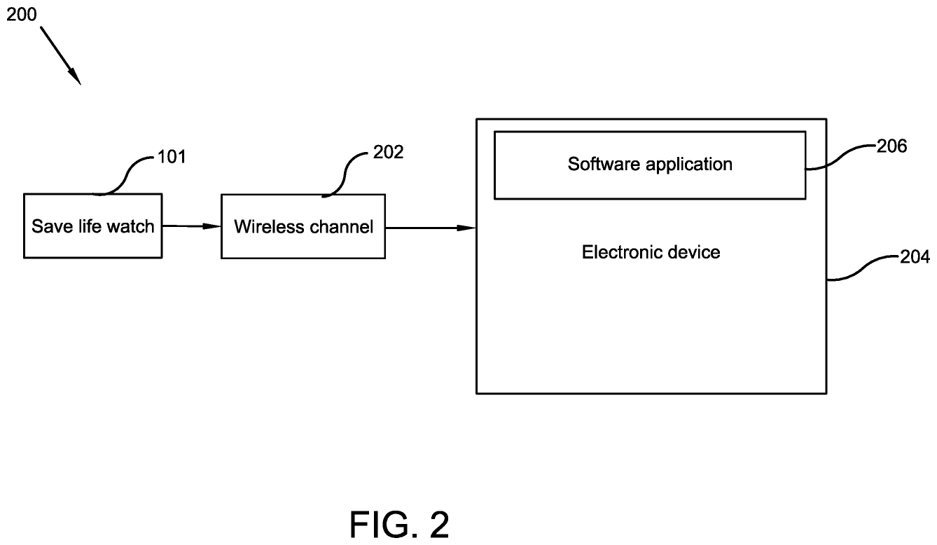

FIG. 2 illustrates a schematic diagram showing connection of the save life watch 101 of the present invention with a companion smartphone application installed on a paired electronic device. As shown in FIG. 2, the save life watch 101 creates an alert generating system 200 in which the watch device 101 can connect to one or more paired smartphone/handheld electronic devices 204 using a wireless channel 202. The electronic device, such as smartphone device 204, has a companion software application 206 that receives alerts and notification from the save life watch 101. One or more sensors of the watch device 101 detect the submerged timing, location and one or more vital signs, such as heart rate etc., of a user and automatically transmit real-time alerts to one or more mobile phones 204, each having an installed software application 206.

When one or more sensors of the watch device 101 breach a predefined threshold value, a corresponding alert is activated and is transmitted to one or more paired electronic devices 204, each having an installed software application 206. Using a processor, the sensors, individually or collectively, detect and determine whether the user is in an emergency situation such as drowning, a medical emergency or has been abducted or is lost, etc., or any other type of emergency situation as is known in the art. The alert can have a user's location which is detected by the GPS module of the watch device 101, information related to time submerged in water which could indicate drowning or respiratory distress, and other vital signs detected by built-in sensors on the watch device 101.

It should be appreciated that parent, guardians, care takers, etc., can receive real-time alerts from the user. Further, the location sensor of the watch device 101 is useful in finding users that have gotten lost, such as small children, or those with dementia. The alert or notification shown on the software application 206 can be in the form of push notification, sound alert or a visual alert, etc., or any other suitable notification as is known in the art.

The wireless channel 202 may include a data network such as, but not limited to, an Internet, a Local Area Network (LAN), a Wide Area Network (WAN), a Metropolitan Area Network (MAN), etc., or any other suitable data network as is known in the art. In some embodiments of the present invention, the wireless channel 202 may include a wireless network, such as, but not limited to, a cellular network, and may employ various technologies including an Enhanced Data Rates for Global Evolution (EDGE), a General Packet Radio Service (GPRS), etc., or any other suitable technologies as are known in the art. In some embodiments of the present invention, the wireless network 202 may include or otherwise cover networks or sub-networks, each of which may include, for example, a wired or a wireless data pathway. The watch device 101 can also communicate data using any cellular protocol, ZigBee protocol, Bluetooth protocol, Wi-Fi protocol, WiMAX protocol, USB protocol or ultrawideband protocol. Embodiments of the present invention are intended to include or otherwise cover any type of communication mediums, including known, related art and/or later developed technologies as are known in the art.

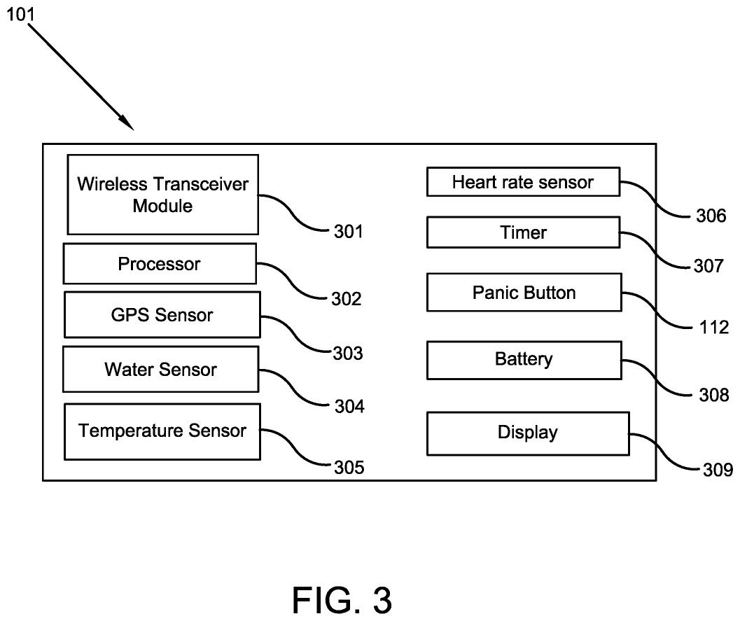

FIG. 3 illustrates a block diagram showing exemplary components and sensors disposed in the smart wearable watch device 101 of the present invention. As stated supra, the smart wearable watch device 101 has a plurality of electronic components and sensors for continuously measuring vital signs, location, duration in water and other related information of the user. The watch device 101 is further configured to transmit all the measured information and any variations from predefined threshold values in the form of alerts to paired smartphones for security purposes. More specifically, a wireless transceiver module 301 functions as a wireless transceiver, allowing the save life watch device 101 to connect to one or more nearby smartphone devices. The wireless transceiver module 301 allows the save life watch device 101 to transmit alerts and notifications to one or more paired electronic devices, which are displayed in the companion software application.

The save life watch device 101 also has a processor 302 for processing measurement signals obtained from the plurality of sensors. The processor 302 also digitizes the analog signals for display and for transmission as digital signals, in case analog sensors are utilized for the measurement of vitals. The processor 302 can also perform some rudimentary processing of the sensor data, if desired, for deciding which alerts and other information should be transmitted from the wearable watch device 101. For example, the processor 302 can determine if the detected heart rate is less than a minimum threshold value or more than a maximum threshold value. The on-board processor 302 can analyze the time-dependent measurements to generate statistics of a user's blood pressure, such as average pressures and beat-to-beat pressure variations.

The wearable safety watch device 101 also has a GPS sensor 303 that can be used for detecting the exact location of the user, and which helps in identifying the location of the user if he or she become lost. It should be appreciated that the GPS sensor 303 of the watch device 101 is particularly useful to track and locate lost users quickly, such as small children, adults with disabilities or those abducted by a stranger.

A water sensor 304 is used for detecting the depth of the watch device 101 within a body of water, and also for measuring the time that the watch device 101 remains within a body of water. The water sensor 304 can detect the depth of the user underwater, and an alert is activated that emits a high-frequency pitch noise from the watch device 101 if the water sensor 304 detects a depth that is more than a preconfigured depth, or is for a time greater than a pre-defined time. The water sensor 304 also sends an alert to the paired smartphone application and mimics the alert from the paired smartphone application, which indicates that the user is in a potential drowning situation.

A temperature sensor 305 is used for measuring the body temperature of the user, and can instruct the processor 302 to generate an alert if the temperature of the user is outside the normal range.

A heart rate sensor 306 measures the heart rate in Beats per Minute (BPM), using an optical LED light source and an LED light sensor. The light emitted from the light source shines through the skin of the user, and the sensor measures the amount of light that is reflected back. The light reflections are varied as blood pulses under the skin of the user past the light. The variations in the light reflections are interpreted as heartbeats by the watch device 101. The heart rate sensor 306 can detect a user's heart rate when the user is running, walking or exercising in the park, or when the user is in a dangerous situation, such as drowning or in an accident. The heart rate sensor 306 also helps in detecting sudden gasping of breath, abnormally increased or decreased heart rates or the cessation of breathing, any of which can indicate an abnormal heart rate situation. In all such cases, the watch device 101 generates an alert or notification and transmits the alert to the paired smartphone application. Thus, the wearable watch device 101 provides an in-depth, immediate and cost-effective mechanism to evaluate a user's cardiac condition. Certain cardiac conditions can be controlled, and in some cases predicted, before they occur. Moreover, data from the user can be collected and analyzed while the user participates in his or her normal, day-to-day activities.

The watch device 101 has an internal timer 307 that triggers or starts when the watch device 101 is submerged underwater. When the timer 307 exceeds a preconfigured time threshold, such as ten seconds for example, the watch device 101 automatically transmits a real-time alert or notification to one or more nearby paired electronic devices, each having the installed software application. If the timer 307 does not exceed the extended period of time, no alerts or notifications will be generated to the mobile phones. However, vital sign information such as temperature or heart rate can be transmitted by the watch device 101.

The panic button 112 can be pushed by a user when the user finds himself in a dangerous or stressful situation, such as abducted by a stranger. The panic button 112, when pressed, immediately generates an alert or notification to the paired smartphone application, along with a sound alert. Also, the current location of the user is transmitted, thereby allowing others to find the user conveniently.

The watch device 101 has a battery 308 that is used for providing power to the components of the watch device 101. The battery can be in the form of a piezoelectric transducer, or any other suitable battery as is known in the art. The watch device 101 has a touchscreen display 309 configured to display the current time, vital sign information such as heart rate information and other data, etc.

The display 309 can be an analog display or a digital display. Such sensors disclosed above can have embedded batteries, collect power from RF signals or temperature differentials or obtain power from any of a number of additional alternatives as are known in the art. The watch device 101 can automatically start once the user has put the watch device 101 on his or her wrist, such that a user is not required to manually start the watch device 101. In a further embodiment, the watch device 101 can also comprise a camera 310 that can be used to take photos or record events (e.g., a kidnapping).

Furthermore, an advantage might be obtained by utilizing more than one sensor of the watch device 101 to provide further data for analysis. By utilizing a plurality of the above-identified sensors, the determination of a dangerous situation for the user can be more accurate. The plurality of the above sensors notifies a parent or guardian that the user is in danger, such as a potential drowning, or other accident.

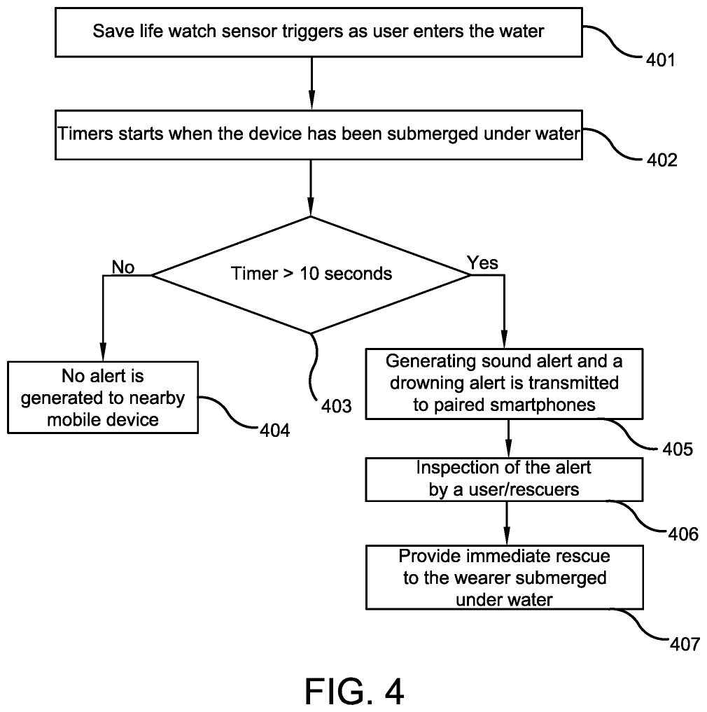

FIG. 4 illustrates a flow diagram showing the operation of detecting a drowning condition and generating a drowning alert by the wearable smartwatch device of the present invention. As shown in FIG. 4, initially at 401, the water sensor of the save life watch device triggers as a user enters a pool, lake or other body of water. The save life watch device is designed to alert others if the user is drowning or struggling in a pool or other body of water. At 402, the save life watch device senses and automatically starts the timer when the watch device is submerged underwater. Then at 403, it is determined by the processor of the watch device whether the watch device has been submerged continuously in water for an extended period of time for example, more than ten seconds. If it is determined that the watch device has not been submerged underwater for than ten seconds, then at 404 no alert or notification is generated by the save a life watch device.

Further, at 403, if it is determined that the time for which the watch device has been submerged under water exceeds the extended period of time, (i.e. more than ten seconds), then at 405, a real-time sound alert with high frequency pitch noise is generated by the watch device to alert nearby people, and also a drowning alert is generated and transmitted to the one or more paired smartphones, each having installed the software application. The alert can be received as a push notification, a text message or an audio message, and at 406 the alert is seen and reviewed by one or more of parents, caregivers, lifeguards or swimming coaches having the paired electronic devices. The alert can also show a user's exact location and the amount of time he or she has been in the water. Then at 407, parents, caregivers, lifeguards, swimming coaches, etc., based on the received alerts, can provide immediate rescue to the user submerged underwater.

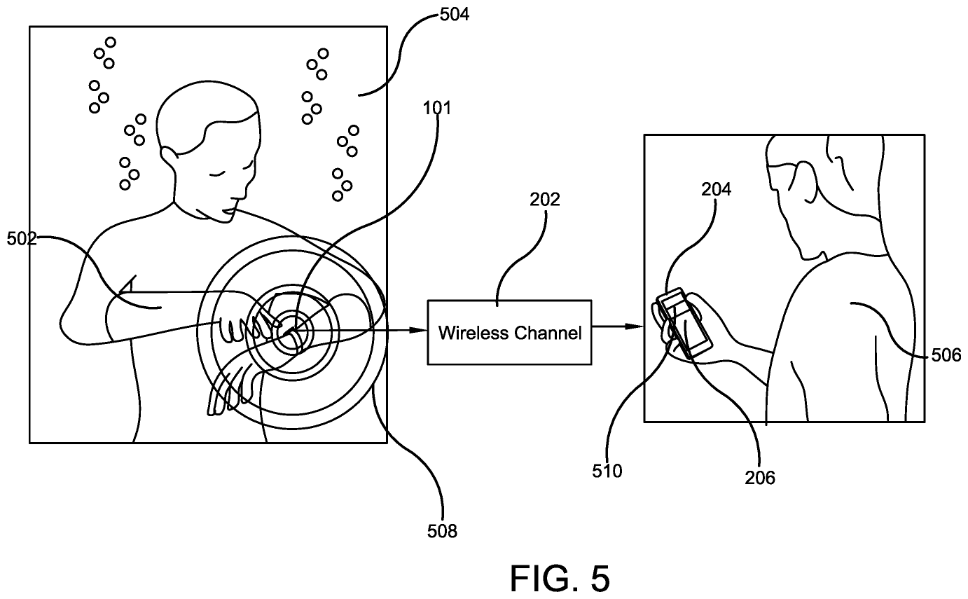

FIG. 5 illustrates a perspective view showing a notification sent from the wearable smartwatch device 101 worn by a child 502 in a body of water 504. As shown in FIG. 5, a young child 502 wearing the safe life watch device 101 is potentially drowning in a body of water 504, such as a swimming pool. As stated earlier, the save life watch device 101 is a wearable device in the form of a flexible wristwatch 101 that is designed to alert others, such as family or friends 506, when the user 502 is drowning or struggling in a pool, lake or other body of water 504. As the child 502 is submerged in water, the timer disposed on the watch device 101 senses the time that the device 101 is submerged, and then accordingly generates an alert that emits a high-frequency pitch noise 508, and sends an alert 510 via a wireless channel 202 to a paired mobile phone 204 having the software application 206 installed. The alert 510 can also include the user's exact location and the amount of time he or she has been in the water.

The watch device 101 prevents a child 502 or other individual from drowning, by ensuring that others 506 are alerted to the user's location if the user is wearing the watch device 101 and becomes submerged in water for a certain period of time. The watch device 101 allows parents and caregivers to know the state of the user quickly, and ensures that the user may be saved from drowning.

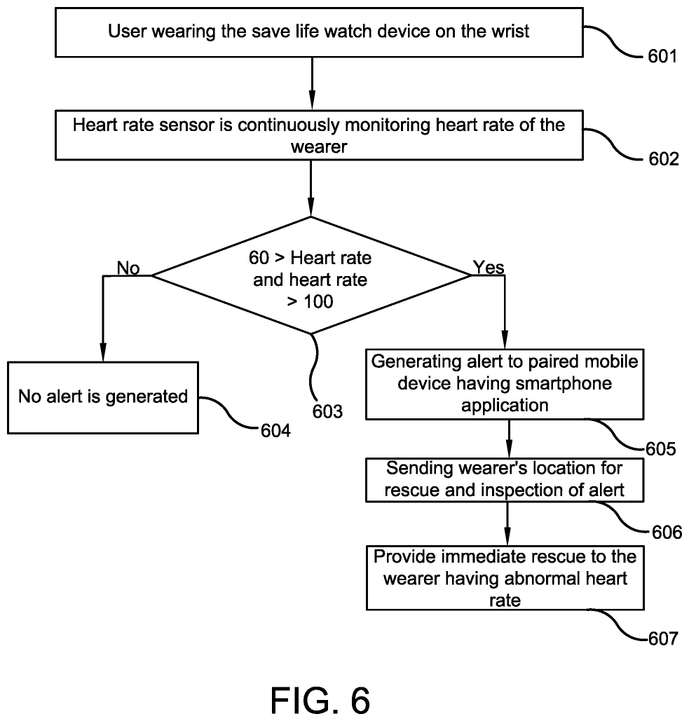

FIG. 6 illustrates a flow diagram showing the generation of an emergency alert by the save life watch device 101 of the present invention, when an abnormal heart rate is detected. Initially, at 601, a user wears the safety wearable watch device on his or her wrist and the sensors disposed within the watch device start monitoring vital signs such as heart rate, location, temperature, etc. Specifically, the wearable safety device includes a heart rate sensor that continuously monitors and displays the heart rate of a user. At 602, the heart rate sensor monitors a user's heart rate when the user is running, exercising or walking, or when the user is in a dangerous situation, such as drowning, an accident, etc. While monitoring, the heart rate sensor in combination with the processor of the watch device, at 603, determines if the detected heart rate is less than 60 beats per minute, or if the user has a heart rate greater than 100 beats per minute. If the detected heart rate is within the range from 60 to 100 beats per minute, then at 604 no alert or notification is generated.

Further, if the heart rate is less than 60 beats per minute, or greater than 100 beats per minute for at least several minutes, then at 605, a real-time alert is generated and transmitted to all paired electronic devices. The alert can show the current and historic heart rate and exact location of the user on the display of the mobile phone. The software application can also display one or more possible reasons for the variable heart rate. At 606, the received alert is seen and reviewed by one or more parents, guardians, caregivers or family members having a paired electronic device. Then at 607, based on the received alerts, the parents, guardians, caregivers or family members can provide immediate rescue to the user having the abnormal heart rate.

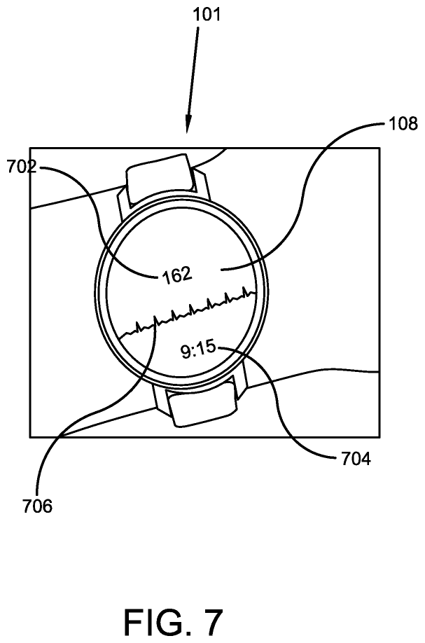

FIG. 7 illustrates an exemplary view of the information displayed on the save life watch device of the present invention. The digital dial display 108 displays a user's heart rate 702 in real-time. The digital dial display 108 also displays the current time 704. As stated, the watch device 101 has a heart rate sensor that based on the measurement of a user's pulse waves via emitting red or infrared light from a body surface, detects the change in blood flow and displays the heart rate 702 in real-time and in a numerical value, and also optionally in graphical form 706. It should be appreciated that the information displayed on the display 108 can be customized by the user based on the user's needs and/or wants.

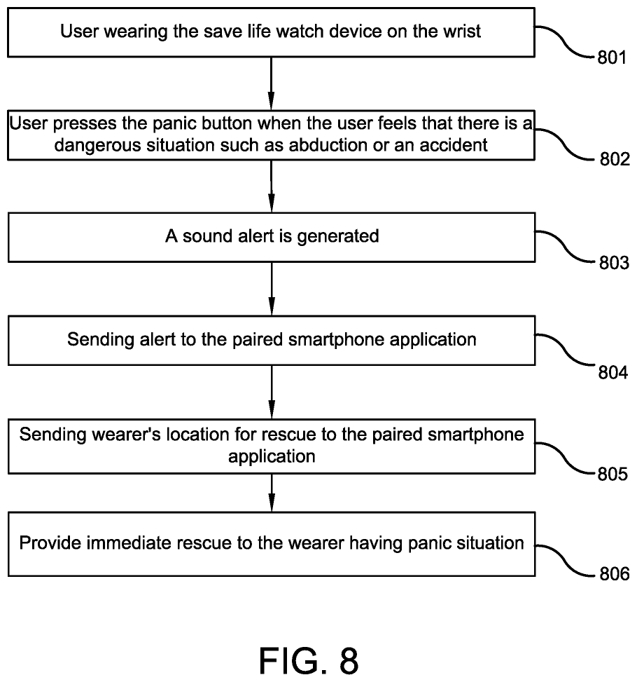

FIG. 8 illustrates a flow diagram showing an alert generated and transmitted by the save life watch device of the present invention when a user pushes the panic button. As shown in FIG. 8, initially at 801 a user wears the save a life watch device on his or her wrist with all the activated sensors. While wearing the safe life watch device, the user detects that there is a dangerous situation, emergency or accident, etc., or any other type of situation needing immediate help. At 802, the user presses the panic button integrated on the save life watch device. At 803, as the user presses the panic button, and a loud, audible sound alert is generated. At 804, the watch device also sends an emergency alert to one or more paired smartphones for immediate rescue. Optionally, the safe life watch device has the capability of alerting emergency services such as 911, police or medical aid services when the panic button is pushed. At 805, the alert states a user's exact location to enable other users to track and locate the user. Then at 806, based on the received alerts, the parents, guardians, caregivers or family members can provide immediate rescue to the user.

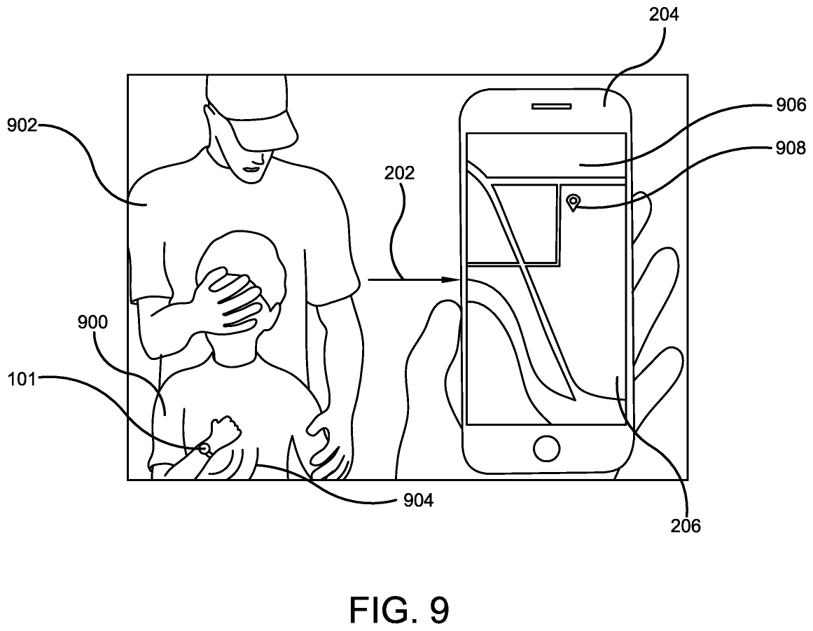

FIG. 9 illustrates a perspective view showing the use of the save a life watch device of the present invention during a child abduction. As shown in FIG. 9, the child 900 is wearing the save a life watch device 101. When the child 900 feels that there is a dangerous situation, such as an abductor 902 trying to abduct the child 900, then the child 900 presses the panic button integrated in the watch device 101. When the child 900 presses the panic button, the watch device 101 generates a sound alert 904 and sends an alert 906 to the mobile device 204 having the software application 206 installed. The alert 906 is transmitted via a wireless communication medium 202 to the mobile device 204. The alert 906 shows parents, guardians, caregivers, etc., the exact location 908 of the user 900 in the form of a digital map. The parents, guardians or caregivers upon receiving the alert 906 on the mobile device 204, can provide immediate rescue to the child 900.

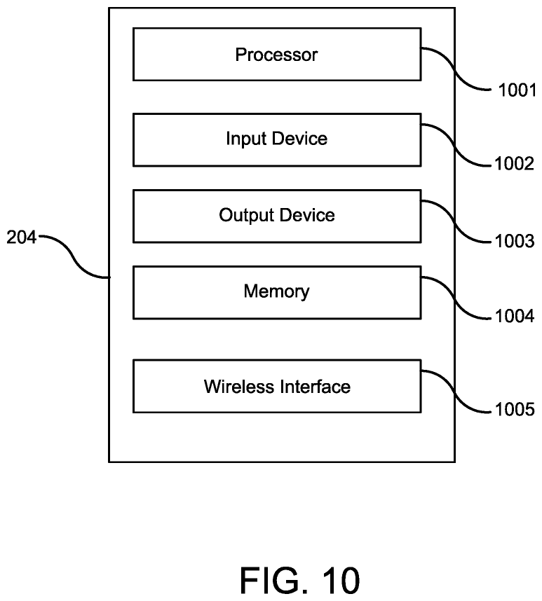

FIG. 10 illustrates an exemplary electronic device 204 in which the software application that communicates with the save life watch device 101 is installed. The electronic device 204 includes several components, such as a processor 1001 configured to perform one or more functions described herein, in accordance with the computer-implemented instructions of the software application 206. Further, the electronic device 204 includes an input device(s) 1002, such as a mouse, keyboard, touch input device, voice input device, etc., for entering data and information. The electronic device 204 also includes one or more output device(s) 1003, such as a monitor, presence-sensitive display or other display device, etc. The display, which can be a touch interface, acts as both an input device 1002 and an output device 1003. The display may be, for example, a liquid crystal display (LCD), a light emitting diode (LED) display, an organic light emitting diode (OLED) display, an active OLED (AMOLED), Micro-electromechanical systems (MEMS) display or electronic paper display, etc. The display can show, for example, various contents (i.e., text, images, videos, icons and/or symbols, etc.) to the user. The display can include a touch screen, and may receive, for example, a touch, gesture, proximity or hovering input using an electronic pen or a part of a user's body.

Further, the electronic device 204 has memory 1004 used for storing programs (sequences of instructions) or data (i.e., program state information) on a temporary or permanent basis for use in the computer system. Memory 1004 can be configured for short-term storage of information as volatile memory, and therefore does not retain stored contents if powered off. Examples of volatile memories include random access memories (RAM), dynamic random-access memories (DRAM), static random-access memories (SRAM) and other forms of volatile memories known in the art. The processor 1001, in combination with one or more of the memory 1004, input device(s) 1002 and/or output device(s) 1003, is utilized to provide users with a means to execute instructions on the software application. Finally, the connection to a network is provided via a wireless interface 1005.

Certain aspects of the present disclosure are described above with reference to block and flow diagrams of systems, methods, apparatuses, and/or computer program products according to various implementations. It should be understood that one or more blocks of the block diagrams and flow diagrams, and combinations of blocks in the block diagrams and the flow diagrams, respectively, can be implemented by computer-executable program instructions.

These computer-executable program instructions may be loaded onto a special-purpose computer or other particular machine, a processor or other programmable data processing apparatus to produce a particular machine, such that the instructions that execute on the computer, processor or other programmable data processing apparatus create means for implementing one or more functions specified in the flow diagram block or blocks. These computer program instructions may also be stored in a computer-readable storage media or memory, that can direct a computer or other programmable data processing apparatus to function in a particular manner, such that the instructions stored in the computer-readable storage media produce an article of manufacture, including instruction means, that implement one or more functions specified in the flow diagram block or blocks. As an example, certain implementations may provide for a computer program product comprising a computer-readable storage medium, having a computer-readable program code or program instructions implemented therein. Said computer-readable program code is adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process, such that the instructions that execute on the computer or other programmable apparatus provide elements or steps for implementing the functions specified in the flow diagram block or blocks.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “save life watch device”, “wearable safety device”, “wearable smartwatch device”, “smart wristwatch device”, “watch device”, and “wearable device” are interchangeable and refer to the safety wearable device 101 of the present invention.

Notwithstanding the forgoing, the wearable safety device 101 of the present invention can be of any suitable size and configuration as is known in the art without affecting the overall concept of the invention, provided that it accomplishes the above stated objectives. One of ordinary skill in the art will appreciate that the size, configuration and material of the wearable safety device 101 as shown in FIGS. 1-10 is for illustrative purposes only, and that many other sizes and shapes of the wearable safety device 101 are well within the scope of the present disclosure. Although the dimensions of the wearable safety device 101 are important design parameters for user convenience, the wearable safety device 101 may be of any size that ensures optimal performance during use, and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A safety system configured to alert others if a user is in a dangerous or stressful situation, the safety system comprising:
a paired electronic device comprising a software application; and
a wearable watch device comprising a wrist strap, a watch body, a display, a plurality of sensors and a metal electrode; and
wherein the display is configured to display time, date, and a user's vital information such as heart rate and blood pressure;
wherein a rear surface of the watch body has a metal electrode that allows a heart rate variability to be measured; and
wherein the metal electrode contacts a user's skin to measure vital information; and
wherein the wearable watch device communicates with the paired electronic device via a wireless channel; and
wherein the paired electronic device receives alerts and notification from the wearable watch device based on information received from the plurality of sensors.

2. The safety system of claim 1, wherein the wrist strap is secured to the watch body via at least one wrist buckle.

3. The safety system of claim 1, wherein the watch body comprises a crown for switching digital and analog displays and adjusting time and date on the display.

4. The safety system of claim 1, wherein the watch body comprises a panic button for alerting others of an emergency.

5. The safety system of claim 4, wherein the watch body comprises a speaker for emitting audible sounds.

6. The safety system of claim 5, wherein the watch body comprises push buttons for adjusting settings of the plurality of sensors.

7. The safety system of claim 1, wherein the wearable watch device comprises a wireless transceiver module that allows the wearable watch device to connect with the paired electronic device.

8. The safety system of claim 7, wherein the wearable watch device comprises a processor for processing measurement signals obtained from the plurality of sensors.

9. The safety system of claim 8, wherein the plurality of sensors of the wearable watch device comprises a GPS sensor that can be used for detecting an exact location of the user.

10. The safety system of claim 9, wherein the plurality of sensors of the wearable watch device comprises a water sensor which is used for detecting depth of the wearable watch device within a body of water.

11. The safety system of claim 10, wherein the wearable watch device comprises an internal timer that starts when the wearable watch device is submerged under water.

12. The safety system of claim 11, wherein the plurality of sensors of the wearable watch device comprises a temperature sensor which is used for measuring body temperature of the user.

13. The safety system of claim 12, wherein the plurality of sensors of the wearable watch device comprises a heart rate sensor that measures a user's heart rate in Beats per Minute (BPM) using an optical LED light source and an LED light sensor.

14. The safety system of claim 13, wherein the wearable watch device comprises a battery for providing power to components of the wearable watch device.

15. A method of detecting a drowning condition and generating a drowning alert via a wearable watch device comprising the steps of:
triggering the water sensor of the wearable watch device as a user enters a body of water;
sensing that the wearable watch device is submerged under water;
starting a timer when the wearable watch device is submerged under water;
determining via a processor whether the wearable watch device has been submerged continuously in the body of water for an extended period of time, wherein if it is determined that the wearable watch device has not been submerged for greater than a predetermined threshold, then an alert is not generated, and further wherein if it is determined that the wearable watch device has been submerged for greater than the predetermined threshold, than an alert is generated;
receiving and reviewing the alert by at least one of parents, caregivers, lifeguards, and swimming coaches having a paired electronic device; and
providing immediate rescue to the user submerged under water.

16. The method of claim 15, wherein the alert is an audible alert.

17. The method of claim 16, wherein the alert is a text message sent to a paired electronic device.

18. The method of claim 17, wherein the alert comprises a user's exact location and amount of time the user has been submerged in the body of water.

19. A method of generating an emergency alert via the wearable watch device comprising the steps of:
wearing the wearable watch device on a user's wrist;
monitoring vital signs such as heart rate, location, and temperature of the user via sensors within the wearable watch device;
determining if a detected heart rate is within a predetermined threshold while monitoring the heart rate sensor in combination with the processor of the wearable watch device, wherein if the detected heart rate is within the predetermined threshold, no alert is generated, and further wherein if the detected heart rate is outside of the predetermined threshold, then a real time alert is generated and transmitted to paired electronic devices;
displaying the current heart rate and exact location of the user on the alert;
receiving and reviewing the alert by at least one of parents, guardians, caregivers, or family members having a paired electronic device; and
providing immediate rescue to the user having the abnormal heart rate.

20. The method of claim 19, wherein when a user pushes the panic button, an audible sound is generated, an alert is sent to emergency services, and an alert is sent to at least one of parents, guardians, caregivers, or family members having a paired electronic device.
